(19) United States
(12) Patent Application Publication
(10) Pub. No.: US 2006/0234329 A1
(43) Pub. Date: Oct. 19, 2006

(54) Method for determining the antiradical defense potential and use thereof, in particular in veterinary and human preventive therapeutics

(76) Inventor: Michel Prost, Couternon (FR)

Correspondence Address: OSTROLENK FABER GERB & SOFFEN, 1180 AVENUE OF THE AMERICAS, NEW YORK, NY, 100368403, US

(21) Appl. No.: 11/408,582

(22) Filed: Apr. 20, 2006

Related U.S. Application Data
(63) Continuation of application No. PCT/FR04/02716, filed on Oct. 22, 2004.

(30) Foreign Application Priority Data
Oct. 22, 2003 (FR) 03 12351

(51) Int. Cl. C12Q 1/26

(52) U.S. Cl. 435/25

(57) ABSTRACT

The present invention relates to a method for determining in vitro the global antiradical defense potential of a living organism or a physical, chemical or biological agent. Said method, which comprises the use of free radicals as a means for inducing cellular lysis and the evaluation of said cellular lysis, is characterised in that it comprises hydrolysing a cellular material before or after the release of free radicals from a free-radical generator into the resulting reaction medium. Said method is useful for following-up the health condition of human beings, animals and plants in vitro, managing the stresses thereof and retarding the ageing thereof. The antiradical substances are particularly useful against spongiform encephalitis.

The present invention relates to evaluating the antiradical defences of a living organism. More specifically, it relates to a method for determining the antiradical defence potential of a living organism or the variation in said antiradical potential brought about by physical (such as in particular the X-rays, β, γ and UV radiation), chemical or biochemical means in a living organism, on the one hand, and to the use of this method in particular in preventive human and veterinary therapeutics, on the other hand.

### PRIOR ART

It will be recalled that free radicals are highly reactive oxidising substances, which play an important role in acute phenomena (such as trauma and ischaemia) and are involved in numerous chronic pathological conditions.

Under normal biological conditions, the living organism produces free radicals in vivo and naturally provides the means required for protecting itself from them, principally enzymatic and/or chemical detoxification systems; overall, there is an equilibrium in situ between said free radicals which are produced and said detoxification systems.

However, aggressive factors (of various kinds, in particular UV radiation, stress, pollution, alcoholism, tobacco smoking, etc.) increase in vivo formation of free radicals, which gives rise to pathological states. Indeed, although the living organism is naturally protected against free radicals by said detoxification systems, these systems may be overwhelmed, the break-down of the equilibrium (i.e. their being overwhelmed) being directly related to genetic factors, to the environment, to lifestyle etc., in a nutshell the antiradical defence capacity of said living organism.

It will also be recalled that free radicals exercise their harmful effects:
at the cellular level: peroxidation of the polyunsaturated fatty acids of the phospholipid membranes and hence formation of cytotoxic peroxides resulting in inflammation and cell death; furthermore, oxygenated free-radical intermediates activate carcinogenesis and cardiovascular diseases in particular; and,
at the extracellular level: degradation of the principal constituents and hence modification of the permeability and structure of tissues, in particular of the skin, such deterioration promotes skin ageing and ageing in general.

Factors which provide protection from free radicals essentially fall into two categories:
enzymatic: superoxide dismutase (SOD), catalase and the selenium glutathione peroxidase; and,
chemical: scavengers which block free radicals, on the one hand, and antioxidants (vitamin C, vitamin A, vitamin E, vitamin K, Se, cysteine, methionine, ubiquinone or coenzyme Q) which limit the effects of free radicals, on the other hand.

Granted European Patent EP-B-0418335 discloses a method for evaluating the antioxidant (i.e. antiradical) activity of a chemical or physical agent to be tested by lysing a cellular material, in particular erythrocytes, by free radicals.

According to said method:

(1) a free radical generator is brought into contact, in an appropriate liquid biological medium, with a cellular material (I) selected from the group made up of
(a) human, animal and plant cells,
(b) fragments of said cells, and
(c) synthetic walls containing liposomes,
said cellular material, which comprises a colorant which can be released on lysis by free radicals,
having first been brought into contact with a physical or chemical agent (II);

(2) the release of free radicals from said free radical generator is induced; and,

(3) lysis of the cellular material is evaluated by measuring optical density relative to a control sample containing said cellular material which has not been brought into contact with said physical or chemical agent.

One method which is recommended for evaluating the lysis in stage (3) involves determining the time (T½) taken for lysis of 50% of the cellular material, in particular the cells of said material, and preferably of erythrocytes (c.f. in this respect EP-B-0418335, page 4, lines 52-55).

This known method has been standardised using whole human or animal blood, which is diluted, instead of the above-stated erythrocytes isolated from whole blood. The standardised method has been successfully marketed under the name “KRL Test” (kit radicaux libres [=free radical kit]) in particular for determining (i) the antiradical defence capacity of a human, animal or plant cellular tissue, or (ii) the possible antiradical properties of a physical, chemical or biological agent. More specifically, the KRL test is performed by 1) bringing (a) previously diluted control whole blood into contact in an appropriate buffer medium with (b) an agent to be tested (in particular a cellular material, a substance or a composition) and (c) a free radical generator, 2) inducing release of the free radicals, 3) monitoring the lysis kinetics of the erythrocytes from the control whole blood by measuring the absorbance of the reaction medium, then 4) comparing said latter value with that of control whole blood determined under the same conditions without the agent to be tested.

As a variant, this standardised method may successfully be used to evaluate the antiradical potential of whole blood from a human or respectively animal subject relative to control whole blood of the same kind.

According to the KRL test, it may be noted that, in a healthy human or animal, there is a mean T½ value which expresses antiradical defence capacity. The recent article by Jean-François LESGARDS et al., published in May 2002 in Environmental Health Perspectives, 110 (no. 5), pages 1 to 9, confirms that this mean value is or falls within a mean range. Below this range, the tested human, animal or plant subject is deemed to be in a pathological or prepathological state by virtue of inadequate antiradical defence.

Despite the effectiveness of the KRL test, certain anomalies on whole blood have been observed in some specific cases: while T½ values of below normal (i.e. below the mean normal range, such as is the case with erythrocytes) were expected, it proved possible to achieve values which were either identical to or greater than said normal value.

Thus, in diabetic patients, the T½ value evaluated on whole blood is generally similar to or greater than that of healthy subjects, and in a freshly irradiated onion, antiradical resistance is greater than that of an unirradiated onion. These are the anomalies which it was desired to study and which form the basis of the invention.

Furthermore, it is known from the article by Jan STAGSTED et al., Free Radical Research, 2002, 36 (no. 7), pages 779-789, to monitor the lysis of erythrocytes (isolated from whole blood of humans, cows, pigs, chickens, pheasants, rats or cats) brought about by an oxidising agent, namely H₂O₂ or free radicals [originating from a free radical generator consisting of 2,2′-azobis(2-amidinopropane)dihydrochloride] by measuring the activity of lactate dehydrogenase (LDH) or the content of haemoglobin in the supernatant. More specifically, the parameters monitored here are the reduction in LDH activity or, respectively, the reduction in the content of haemoglobin. However this article, which confirms the Applicant's unpublished results, which have in particular been obtained in horses, cattle, pigs and ducks, neither describes nor suggests the sequence of operations according to the invention, namely: hydrolysis, release of free radicals and evaluation of the lysis brought about by said free radicals, in order to determine overall antiradical resistance.

### BASIS AND OBJECT OF THE INVENTION

The starting point was the hypothesis that a living organism comprises, on the one hand, immediately available antiradical reserves, i.e. that there is an antiradical potential which is directly mobilised against free radical shock or oxidative stress, and, on the other hand, antiradical reserves which are mobilisable (but are not directly accessible on an extemporaneous basis) and which are normally stored by the organism or locked up in the organism, the overall antiradical potential of said living organism being represented by the sum of immediately available antiradical reserves and mobilisable antiradical reserves which are in storage.

The object of the invention is to provide a method which makes it possible to determine antiradical defence potential with the aim of remedying the above-stated anomalies.

It is a further object of the invention to provide a novel technical solution to the problem of screening for, preventing or monitoring pathological or prepathological conditions associated with inadequate resistance to free radicals by humans, animals (in particular warm-blooded animals) and plants.

There is indeed a requirement (a) in humans or animals to prevent the appearance of irreversible pathological conditions such as degenerative neurological diseases; (b) in livestock to screen out (at the latest at the abattoir) those animals likely to constitute a risk to human food and to assess the stress caused by transport and rearing methods; and (c) in plants to monitor the growth and then method of preservation with the aim of optimising the nutritional value of the plants.

One of the objects of the invention is to identify a normal level (or normal range) of antiradical defence by species. Another object is to enable evaluation of a system for regulating defences by species.

### SUBJECT MATTER OF THE INVENTION

The hypothesis stated above has proved to be correct, as is demonstrated and illustrated below. It has consequently been possible to remedy the anomalies of the prior art and to achieve the desired objects.

According to a first aspect of the invention, a method is advocated for the in vitro determination of the overall antiradical defence potential of a living organism or a physical, chemical or biological agent, said method, which involves the use of free radicals as a means for inducing cell lysis followed by evaluation of said cell lysis, being characterised in that it comprises hydrolysis of a cellular material before or during the release, in the resultant reaction medium, of free radicals from a free radical generator.

In particular, a method is recommended for the in vitro determination of the overall antiradical defence potential of a living organism or of a physical, chemical or biological agent, said method, which involves the use of free radicals as a means for inducing cell lysis, being characterised in that it comprises the following steps: (
α) hydrolysis of a sample
   (i) of a first cellular material from a living organism, said first cellular material being a cellular tissue, cells or a cell fragment, or, respectively,
   (ii) of a second cellular material associated with a physical, chemical or biological agent, said second cellular material being a reference product which is a cellular tissue, cells, a cell fragment or a synthetic wall containing liposomes,
(β) during or after said hydrolysis, bringing said sample into contact with a free radical generator,
(γ) inducing release of the free radicals from said free radical generator, and
(δ) monitoring lysis, by optical measurement, of the first or, respectively, second cellular material, relative to a control sample, in order to assess the overall antiradical potential of said living organism or, respectively, of said physical, chemical or biological agent to be tested.

Optical measurement is here carried out by means of a spectrometer. The optical density or absorbance of the test medium is here advantageously measured.

As a variant, in order to assess the antiradical resistance of a physical, chemical or biological agent, said second cellular material may be replaced by a colorant which is degradable by free radicals. The corresponding method is characterised in that it comprises the following steps:
(α) hydrolysis of a sample
of a colorant which is degradable by free radicals and which is associated with a physical, chemical or biological agent,
(β) during or after said hydrolysis, bringing said sample into contact with a free radical generator,
(γ) inducing release of the free radicals from said free radical generator, and
(δ) monitoring the lysis, by optical measurement, of said colorant relative to a control sample, in order to assess the overall antiradical potential of said sample containing said physical, chemical or biological agent to be tested.

According to a second aspect of the invention, it is proposed to use this method for the in vitro screening, prevention or monitoring of pathological conditions and prepathological states of the living organism associated with an abnormal antiradical defence potential.

From this perspective, such use is in particular advocated in respect of degenerative neurological diseases, in particular:

spongiform encephalitis, whether bovine spongiform encephalitis (BSE or mad cow disease), ovine spongiform encephalitis (OSE or scrapie) or human spongiform encephalitis (CJD or Creutzfeld-Jakob's disease),

Alzheimer's disease, and

Parkinson's disease.

A novel use is finally advocated, which use is characterised in that an antiradical substance is used for the preparation of a medicine intended for preventive use in human or veterinary therapeutics in relation to spongiform encephalitis, Alzheimer's disease and Parkinson's disease.

### BRIEF DESCRIPTION OF THE DRAWINGS

In the appended drawings:

FIG. 1 illustrates the mechanism of the variation in optical density (OD) or absorbance by cell lysis, in this case of blood cells;

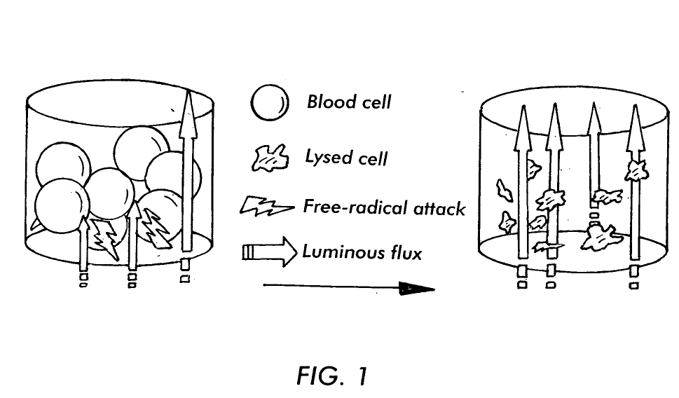

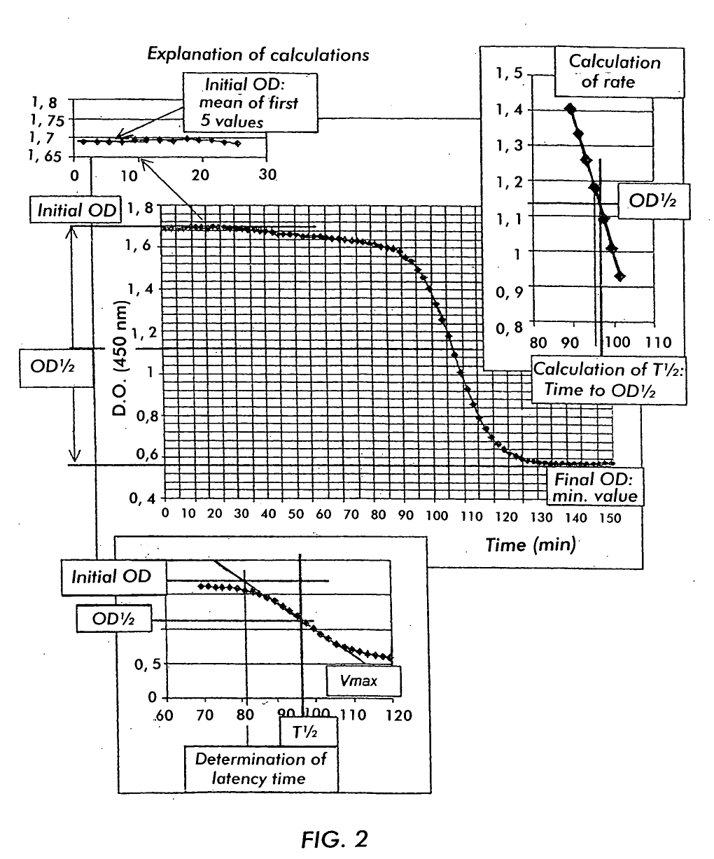

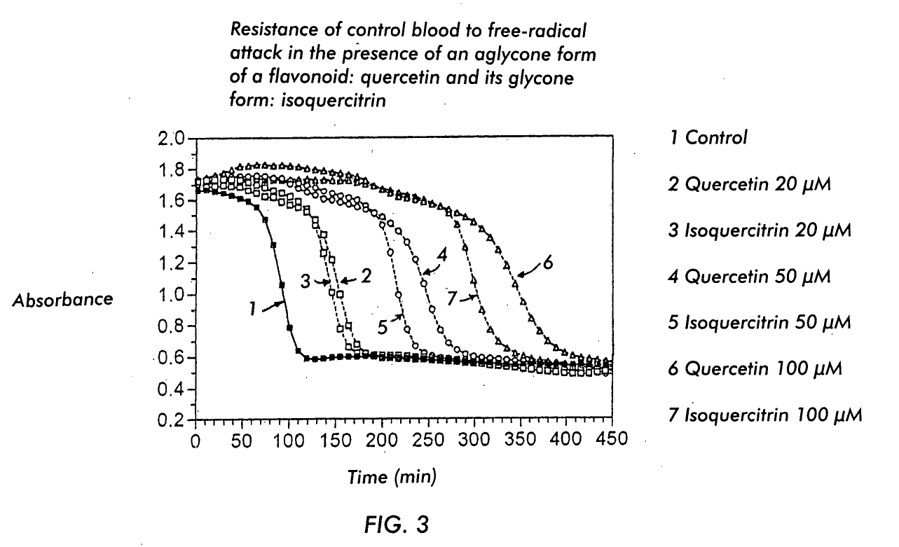

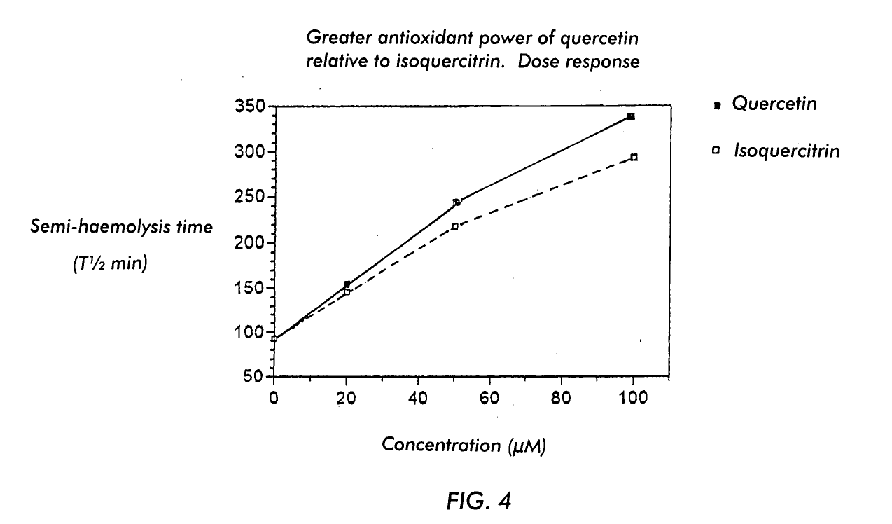

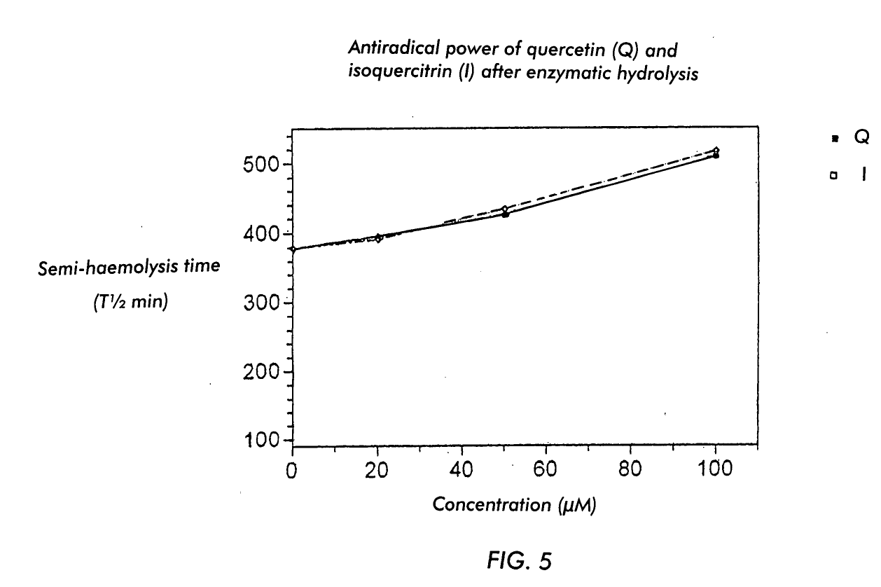

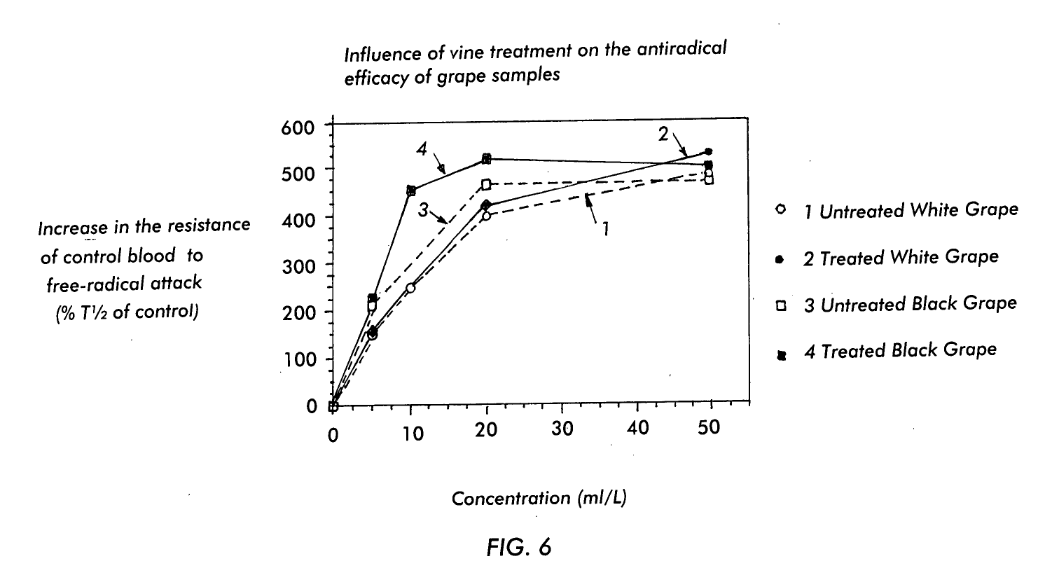

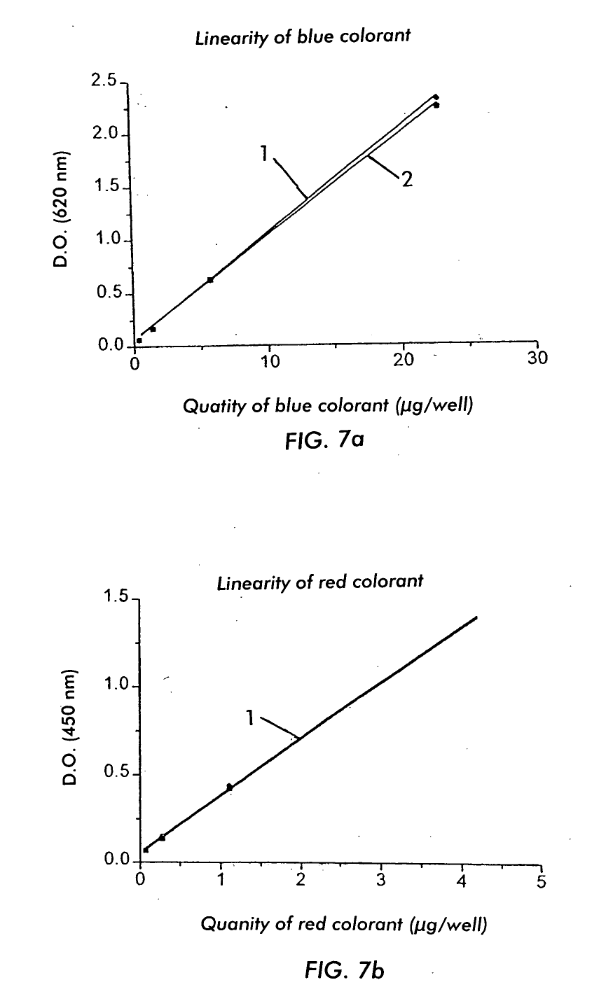

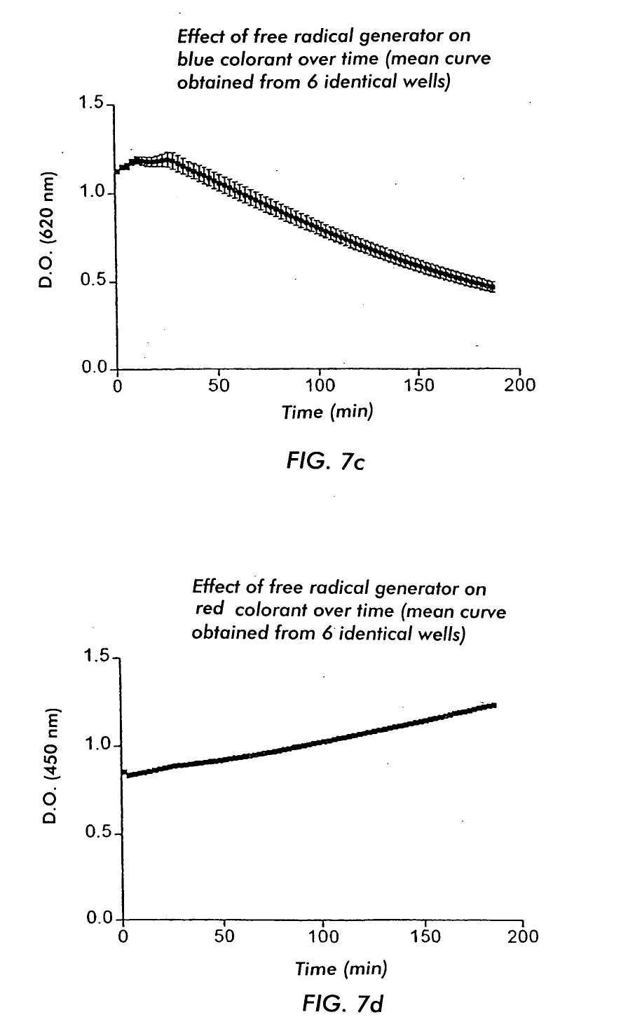

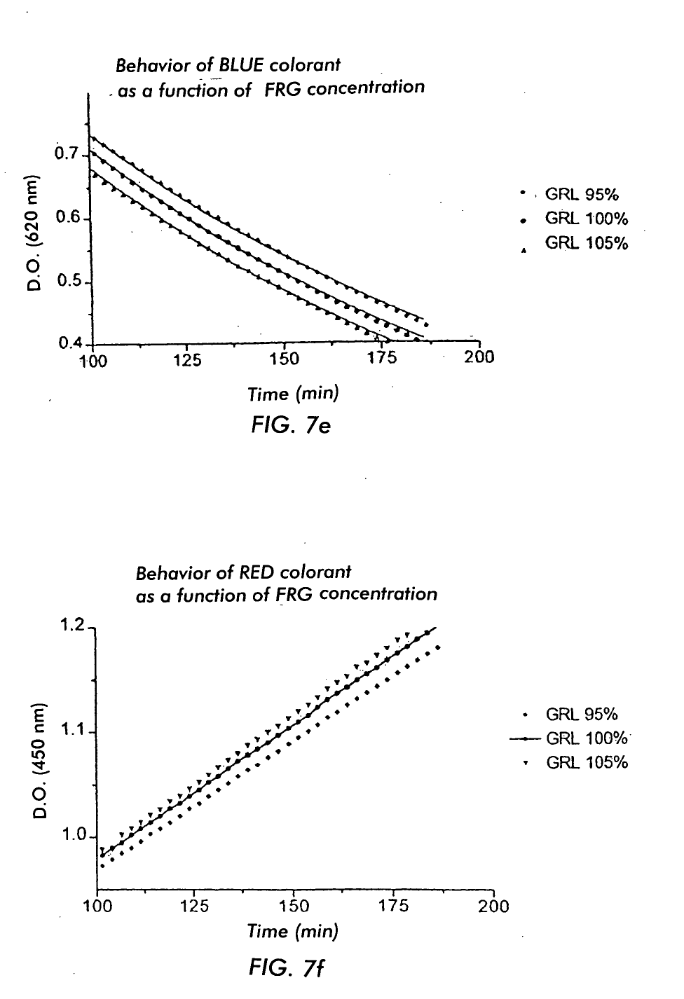

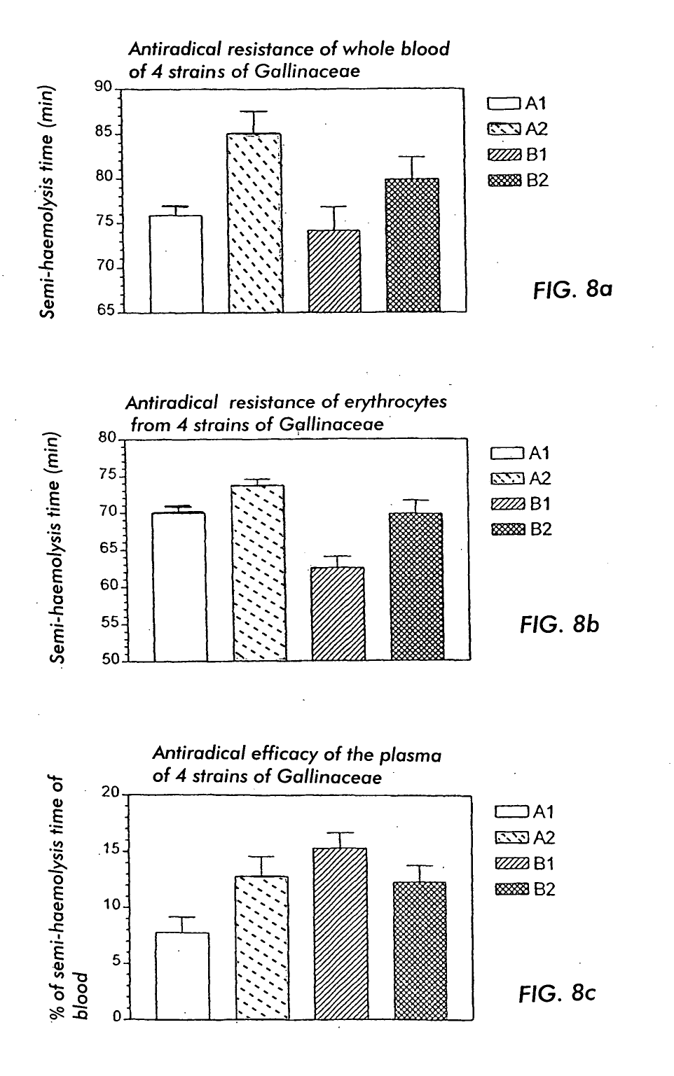

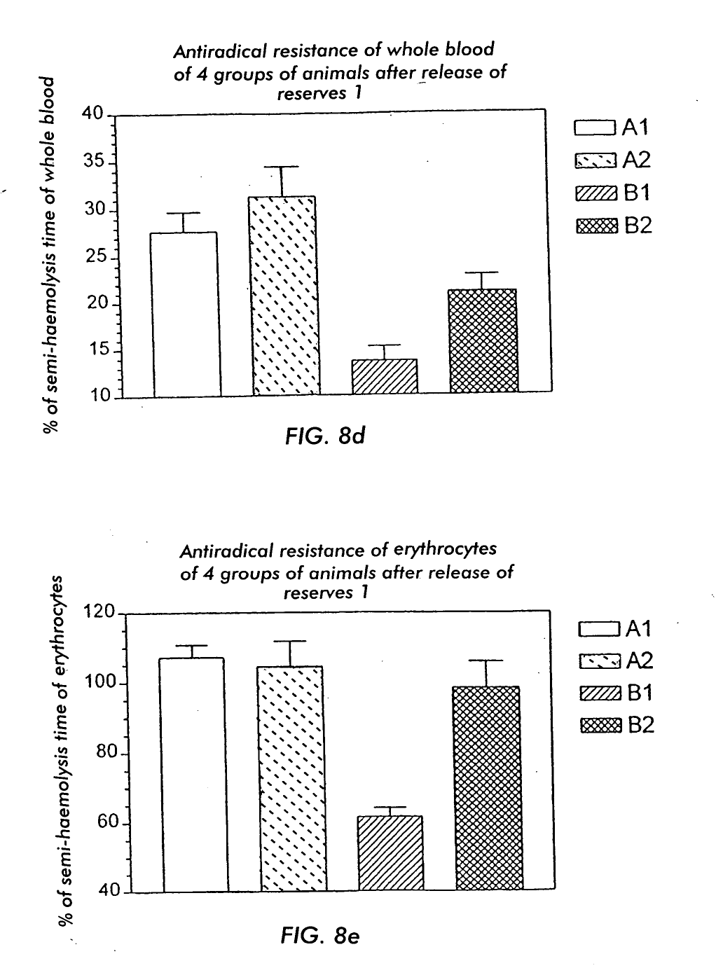

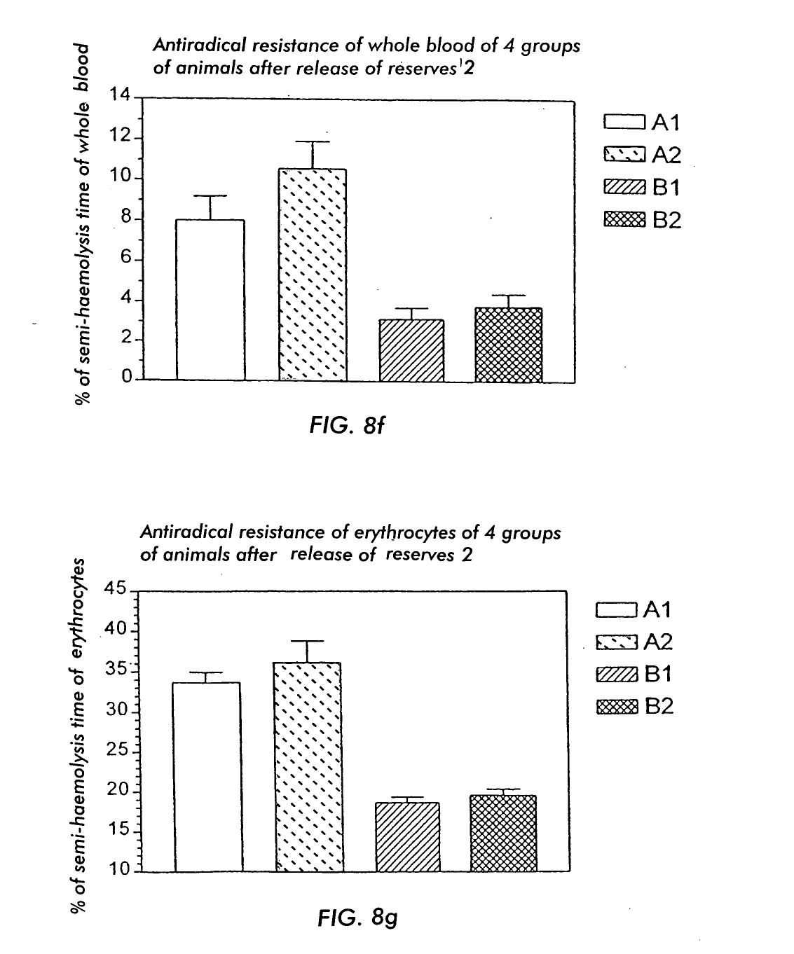

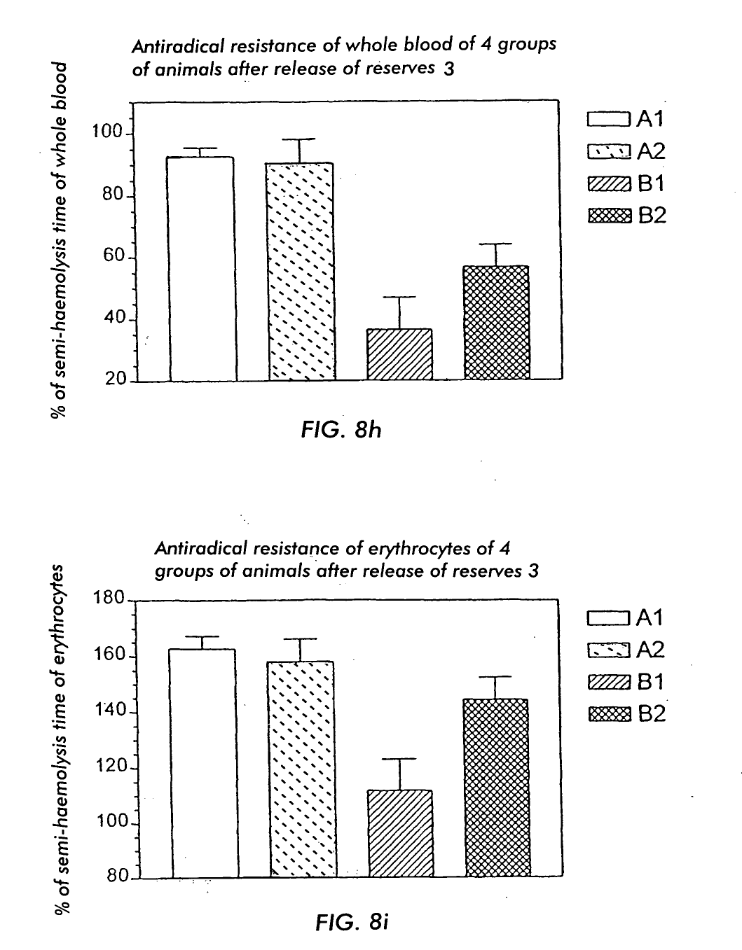

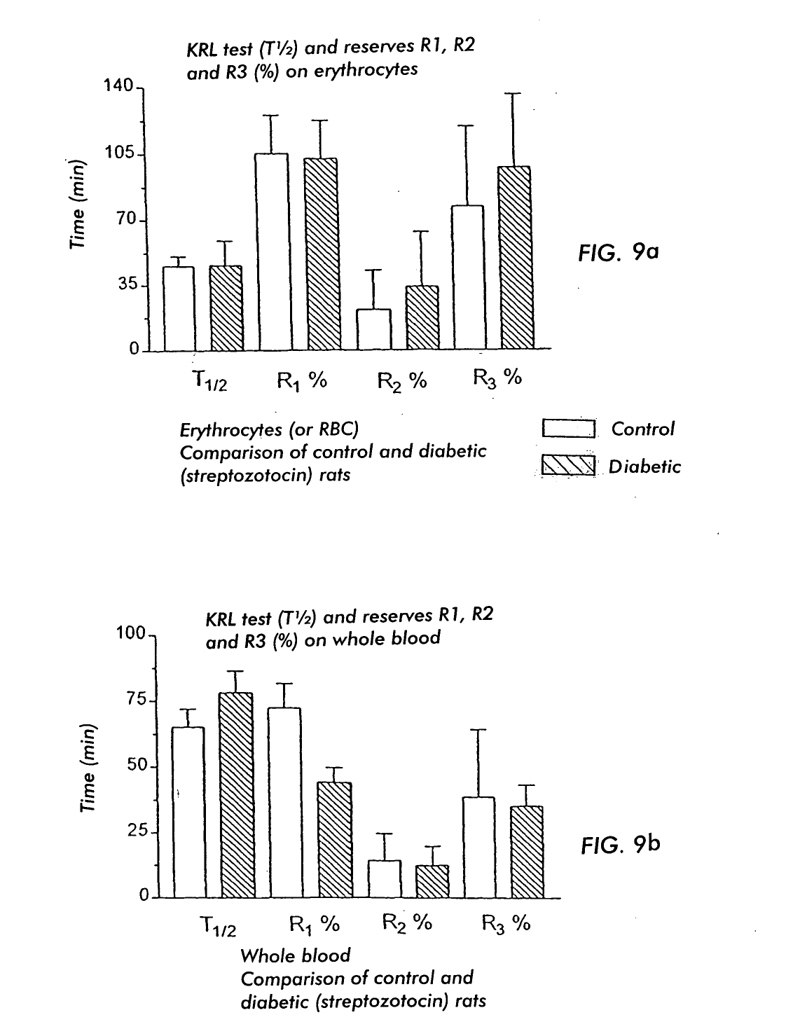

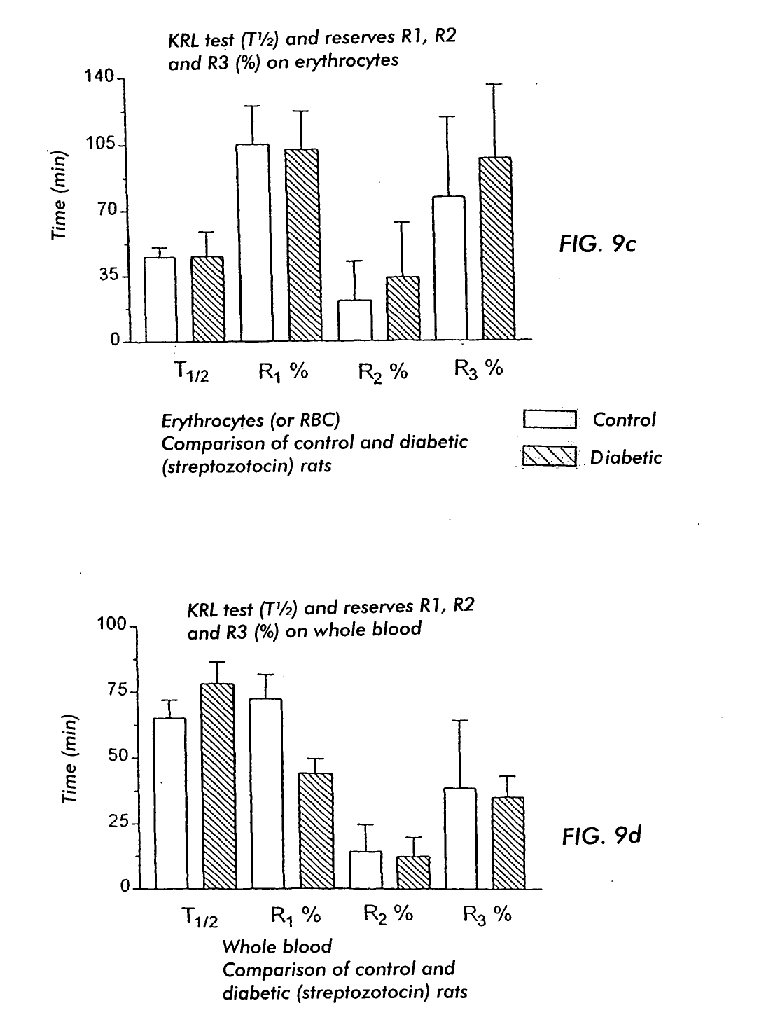

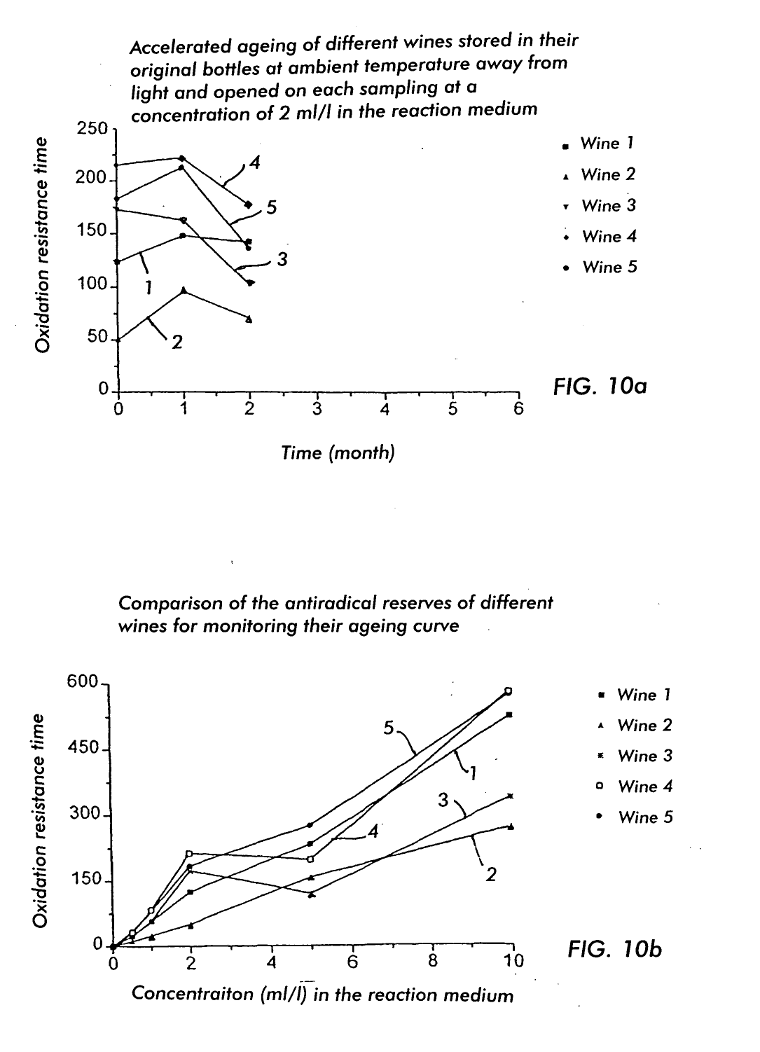

FIG. 2 is a schematic illustration of the method of determining OD½ and the other parameters: the initial OD (mean of the first five values) and the final OD (mean of the last five minimum values) are measured, and the OD½ is obtained therefrom as follows
OD½=0.5×(initial OD−final OD),
the T½ value is calculated which is the OD½ time [the T½ value is the time which corresponds to intersection of the horizontal line passing through OD½ with the curve OD=f(T)]; the maximum rate (Vₘₐₓ) of radical lysis is measured as being the gradient of the curve in the vicinity of its inflection point (OD½, T½), and the latency time (LT, “lag time”) is determined as being the time corresponding to the intersection of the initial OD line with the Vₘₐₓ line;

FIG. 3 shows the absorbance curves=f(T) of quercetin and its glycone homologue, isoquercitrin, at increasing concentrations (0 to 100 μM) without the prior hydrolysis of the invention;

FIG. 4 shows T½=f curves (concentration of quercetin or isoquercitrin) plotted without prior hydrolysis on the basis of the curves of FIG. 3;

FIG. 5 shows the T½=f curves (concentration) of quercetin and isoquercitrin obtained with prior enzymatic hydrolysis; a comparison of the curves of FIG. 5 with those of FIG. 4 reveals the value of prior hydrolysis according to the invention;

FIG. 6 shows the influence of treating vines with a foliar fertilizer relative to untreated vines;

FIGS. 7a and 7b show that, at time T=0 (T₀), the optical density (OD) of two colorants which are degradable under the action of free radicals increases linearly with the concentration of said colorants, whether or not associated with a free radical generator (FRG); in contrast, for a given colorant concentration, FIGS. 7c and 7d show that OD varies as a function of the release over time of the free radicals originating from the FRG; furthermore, FIGS. 7e and 7f show the variation in the OD of said colorants as a function of the concentration of FRG releasing free radicals;

FIGS. 8a to 8i relate to the antiradical resistance of whole blood, of erythrocytes and of plasma of four lines of Gallinaceae (A1, A2, B1 and B2), without release of reserves (KRL test of the prior art) and with release according to the invention of reserves 1 (R1) by hydrolysis by means of a glucosidase, of reserves 2 (R2) by hydrolysis by means of a sulfatase or of reserves 3 (R3) by hydrolysis by means of a glucuronidase;

FIGS. 9a to 9d permit a comparison of the radical resistance of control rats and of rats made diabetic (by administration of streptozotocin), without hydrolysis (KR2 test) and after hydrolysis (evaluation of reserves R1, R2 and R3);

FIGS. 10a and 10b relate to the use of the method according to the invention for monitoring the ageing of wine.

### DETAILED DESCRIPTION OF THE INVENTION

The above-stated anomalies are explained, according to the invention, by abnormal mobilisation of the living organism's antiradical reserves. Thus, in diabetics, (i) the antiradical resistance of whole blood, which is abnormally high, reflects an abnormal mobilisation in the plasma of the antiradical reserves, and (ii), in view of the oxidant stress which develops in said diabetics, the antiradical resistance of the erythrocytes, which are made fragile by glycosylation reactions, is abnormally low.

According to the invention, it is thought that mobilisable antiradical reserves arise in particular:
from polymerisation of antioxidant compounds and/or
from conjugation of said antioxidant compounds with different sugars (for example glucose, rhamnose, glucuronic acid, etc.), alcohols and organic or inorganic acids (such as sulfuric acid), and

that said mobilisable antiradical reserves are stored in the living organism in the form of polymers (in particular as polyphenols), ethers, esters of organic acids and/or sulfates, in particular in the cell walls and especially in the liquid bathing the cells.

In the event of free-radical aggression, these mobilisable antiradical reserves, stored in the organism, are not directly available. They are released by a complex enzymatic mechanism of the “cascade” type, apparently with a system of inhibitors and activators (following the example of the haemostasis cascade), to achieve a final equilibrium. When the equilibrium obtained is not the normal equilibrium, this is indicative of a pathological or prepathological state.

In the method of the invention, the mobilisable antiradical reserves which have been stored are released by hydrolysis. The hydrolysis of step (α) is performed in an appropriate medium, in particular a buffer, at a temperature of between 15 and 60° C.; it is carried out either before the above-stated step (β), or at the same time as step (β). In this latter case, time is saved, but it is then necessary to use a control because said hydrolysis cannot be completed before lysis of the first or second cellular material by the free radicals.

This hydrolysis is (i) enzymatic hydrolysis, (ii) acid or alkaline hydrolysis, or (iii) cleaving by means of a physical agent, in particular radiation.

Enzymatic hydrolysis is advantageously performed for at least 40 minutes (in particular when said hydrolysis proceeds during exposure to the field of free radicals), preferably for 1-3 h and better for 2 h, at a temperature of 20 to 40° C., with a substance selected from among the group made up of osidases (in particular glucosidases, rhamnosidases, glucuronidases, etc.), dealkylases (in particular demethylases), esterases, sulfatases, and mixtures thereof. A mixture of enzymes which is suitable according to the invention may in particular be of the type “glucosidase+demethylase+sulfatase” or “glucosidase+glucuronidase+sulfatase”.

Acid hydrolysis is advantageously performed for at least 40 minutes (in particular when hydrolysis proceeds during exposure to the field of free radicals), preferably for 1-3 h and better for 2 h, at a temperature of 40 to 60° C., preferably at 50° C., and at a pH of less than or equal to 5.5.

Alkaline hydrolysis, which is not essential but instead relates to cleaving ester and lactone functions, is advantageously performed for at least 40 minutes (in particular when hydrolysis proceeds during exposure to the field of free radicals), preferably for 1-3 h and better for 2 h, at a temperature of 50° C., at a pH of greater than or equal to 8.5.

Hydrolysis according to the invention may comprise cleaving by a physical agent such as radiation, for example γ or (better) β radiation, similar to that used for preserving agricultural crops. The physical agent may even be made up of a field of free radicals applied for a sufficient period. This “hydrolysis”, which consists of cleaving ester (i.e. acyl), ether and sulfate groups, on the one hand, or separating the oside fragment from glycones, on the other hand, may therefore involve irradiation of the living organism or of a cellular material originating from said organism.

The cellular material involved in the method of the invention, is selected from the group made up of:
(a) a cellular tissue (i.e. the cells, whether identical or different, plus the liquid or plasma which naturally bathes them or a part of the latter), for example whole blood,
(b) the cells isolated from their environment (and if necessary resuspended in a dilution buffer),
(c) a cell fragment (i.e. a fragment of cell wall, if necessary with the entirety or a proportion of the material contained in the cell, for example lipoproteins), or
(d) a synthetic wall containing liposomes.

The first cellular material which is subject to the hydrolysis of step (a) is advantageously selected from among elements (a), (b) and (c) above, elements (a) and (b) being preferred. Said first cellular material is used for assessing, according to the invention, the overall antiradical potential of a living organism.

The second cellular material is selected from among elements (a), (b), (c) and (d) above, elements (a) and (b) likewise being preferred. It is used for assessing the possible antiradical properties of a physical, chemical or biological agent to be tested, on the one hand, or the overall antiradical potential of a living organism by means of a product originating therefrom (in particular cellular tissue, whole blood, isolated cells, plasma), on the other hand.

The physical, chemical or biological agent is said to be associated with the second cellular material. This means that it has already contaminated the organism from which the second cellular material is collected or that it has been brought into contact, for performance of the present method, with said second cellular material.

The physical agent may be exposure to smoke, to UV or to hazardous radiation.

The chemical agent may be any substance or association of chemical products which it is desired to test in order to determine the possible protection which it may impart to free radicals.

The biological agent may be any substance or association of biological products, for example a plant, an extract of a natural substance, blood plasma or cells which may be different from the cellular tissue or the isolated cells constituting the second cellular material.

As indicated above, the second cellular material may be replaced by a colorant which is degradable by free radicals in order to study the overall antiradical resistance of a physical, chemical or biological agent. Suitable colorants which may be mentioned are in particular oxidoreduction colorants. Good results have been obtained according to the invention with methylene blue (“blue” colorant used hereafter) and oligoanthocyanidin extract (OPC; “red” colorant used hereafter). The colorant used must not be susceptible to the hydrolysis to which the sample containing it is subjected. If, for example, a colorant is susceptible to glucuronidase, it will be used in a method implementing hydrolysis by means of a sulfatase.

As indicated in the above-stated EP-B-0 418 335, preferred free radical generators are those which exhibit kinetics or a reaction rate which is of 0 order (the release of free radicals is constant over time) and better of 1st order (the release of free radicals is linear over time). Oxidising free radical generators which are preferred according to the invention, are, on the one hand, 2,2′-azo-bis(2-amidinopropane)dihydrochloride, which provides 1st order kinetics in an aqueous medium, and on the other hand, 2,2′-azo-bis(2,4-dimethylvaleronitrile) which also provides 1st order kinetics in an oily or organic liquid medium. The free radicals are released from a free radical generator by a method known per se, for example heat, light (in particular light of the visible spectrum or UV light), protons, electrons, X-rays; such release will preferably be initiated by photons, or by heat. For example, adjusting 2,2′-azo-bis(2-amidinopropane)dihydrochloride [abbreviated to AAPH] in an aqueous solution to a temperature of 37° C. is sufficient to initiate release of free radicals according to the reaction:

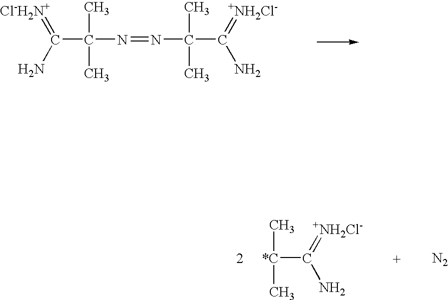

After release of the free radicals has been initiated, monitoring of the lysis of the first or second cellular material is performed optically. More specifically, the variation in optical density (or absorbance) as a function of time is observed and the T½ value (in minutes) is determined which corresponds to lysis of 50% of the cellular material. As indicated above, said cellular material will preferably be a cellular tissue (whole blood for example) or isolated cells.

In practice, optical measurement is performed automatically, for example every 50, 100, 150, 200 and/or 300 seconds, in order to monitor the lysis kinetics of the first or second cellular material. When the variation in absorbance of a test medium (containing for example whole blood or isolated cells) is observed over time starting from the moment that release of the free radicals from the free radical generator (in particular AAPH) is induced, the following are observed:
at the beginning, a period during which absorbance is constant (and relatively high); this is the latency period during which the cells have not been lysed;
as soon as cell lysis begins, a phase during which absorbance falls linearly (the inflection point of the corresponding curve determines the T½ value); then,
when all the cells have been lysed, absorbance is constant (and relatively low).

Thus, when working with whole blood using the prior art KRL test, when the value T½ is rising (see diagram 1), there is:
a range “1” of hypoprotection towards free radicals, which corresponds to a pathological or prepathological state,
a range “N” of normal mean values which may contain “false positive” results, which are abnormally high (as in the case of diabetics) due to abnormal release of antiradical reserves which is not offset by induction of the defences, and
a range “2” of hyperprotection towards free radicals due to abnormal release of a significant part or majority of the mobilisable antiradical reserves, and which corresponds to a pathological or prepathological state because the organism is then drawing on said reserves:

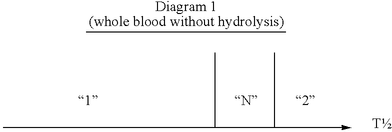

On the other hand, according to the invention, since the overall antiradical potential is evaluated by means of hydrolysis, when T½ is rising (see diagram 2) there is:
an abnormal range “A” of hypoprotection towards free radicals, which corresponds to a pathological or prepathological state or
a range “Nh” of normal mean values, which contains only the results from healthy subjects (a value of T½ beyond said range “Nh” would indicate a procedural error):

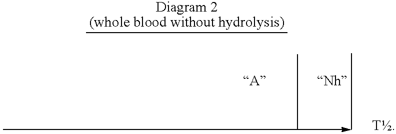

In a sportsperson, for example, the normal range Nh is wider than in the non-sportsperson due to the induction of in particular enzymatic defences brought about by repeated effort; even if the antiradical defence reduces transiently during effort, it remains controlled within the range of normal values, unlike in the non-sportsperson (or the individual who practices sport occasionally), who, when subjected to effort testing, will be outside the control range, so giving rise to the risk of cardiovascular accident which is well-known to cardiologists.

Consequently, the method of the invention makes it possible to avoid any doubt when the T½ value is in the “Nh” range of normal mean values and so to eliminate the above-mentioned anomalies.

Apart from the T½ value, it is also subsidiarily possible to measure, as indicated above, the latency time, the rate of cell lysis by free radicals (i.e. the gradient of the curve) together with the area under the curve, in order to obtain additional parameters for assessment.

Overall antiradical potential may also be stated in EAR units (“Efficacité Anti-Radicalaire” [“Anti-Radical Efficacy”]), 1 EAR unit corresponding to the antiradical power of one millimole of Trolox® per litre of blood, Trolox® (Hoffmann-La Roche Inc.) being a water-soluble equivalent of vitamin E, used as a reference antioxidant, having the structural formula: 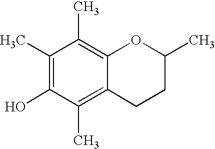
and, in accordance with systematic nomenclature, known as 3,4-dihydro-2,5,7,8-tetramethyl-2H-1-benzopyran-6-ol.

For 1 litre of blood in a healthy man, the following applies:
1 EAR=T½_{w or e}×dilution_{w or e}×concentrationₜ (in mM)×1/ΔT½ₜ
where “w or e” respectively denote whole blood or erythrocytes, and “t” denotes Trolox.

The method of the invention is standardised by using a system which is previously incubated at the test temperature (37° C.), comprising:
a macroplate comprising dilution wells,
a microplate comprising a plurality of analysis wells (in particular 96 wells), and
a spectrometer for measuring the OD or absorbance of light passing through the microplate wells from the bottom upwards.

The three preferred modes for implementing the method of the invention are as follows:
mode I, determination of overall antiradical potential on whole blood from humans or warm-blooded animals (in particular mammals),
mode II, determination of overall antiradical potential on human or animal erythrocytes, and
mode (III), determination on plasma.

Furthermore, when fresh brain tissue from healthy cattle, which contained no insoluble pathogenic prion PrPsc, but did contain non-pathogenic soluble prion PrPc, was subjected to the action of free radicals, the insoluble pathogenic prion was observed to appear in said brain tissue. This formation of PrPsc was revealed by using the technique described in the article by Sabrina CAPELLARI et al., J. Biol. Chem., 1999: 274 (no. 49), pages 34846-34850, this article indicating that PrPc plays a part in the redox equilibrium by preventing or countering oxidative damage.

It was thus unexpectedly observed that fresh brain tissue from a healthy cow produces the pathogenic prion of the PrPsc form under the action of free radicals originating from a free radical generator.

On the basis of this observation, it was deduced that degenerative neurological diseases, and in particular BSE, OSE and CJD, are essentially autoimmune diseases which probably involve two possible routes of infection:
(a) the PrPsc form is inadvertently administered to a human or animal subject, and/or
(b) the PrPsc form is generated in vivo under the action of free radicals which may be produced by the organism,
   in both cases, the organism, which recognises the PrPsc form as being foreign, generates free radicals, via the immune system, to combat the PrPsc. It is these free radicals generated in this manner which will convert the PrPs protein into the pathogenic protein isoform PrPsc, and produce an autoimmune response which explains the phenomenon of pathogenic prion “multiplication”.

An identical or analogous mechanism occurs (it would appear) in the appearance of other degenerative neurological diseases such as Alzheimer's disease and Parkinson's disease. The immune system attacks the proteins which have been modified under the action of the free radicals and have been recognised as enemies. Since these modified proteins are irreversibly stable, the immune system produces free radicals which attack the normal proteins due to their related structures. A chain reaction then occurs.

A novel therapeutic use of antiradical substances for preventing degenerative neurological diseases is thus advocated.

From this perspective, a novel use of an antiradical substance is provided for the preparation of a medicine, said use being characterised in that an antiradical substance is used for the preparation of a medicine intended for use in human or veterinary therapeutics against spongiform encephalitis, in particular bovine spongiform encephalitis (BSE), ovine spongiform encephalitis (OSE) and human spongiform encephalitis (CJD).

For the purposes of this novel use, spongiform encephalitis is prevented by administration of a human or veterinary therapeutic composition comprising at least one antiradical substance in association with a pharmaceutically acceptable vehicle.

These antiradical substances are principally polyphenols, the glycones derived therefrom, and the sulfate derivatives thereof. Among these substances, sulfated ferulic acid (I) may in particular be mentioned. This product may be hydrolysed by two distinct routes shown below in scheme 3.

Scheme 3 shows that cleaving by a sulfatase according to 1 gives rise to trans-ferulic acid (II), whereas cleaving by a demethylase according to 2 ultimately gives rise to trans-caffeic acid (III), via sulfated trans-caffeic acid (IV). Sulfated ferulic acid (I) consequently comprises a double antiradical reserve which is usable by hydrolysis. The antiradical activity of these products was determined according to the prior KRL test without hydrolysis, on the one hand, and according to the invention after hydrolysis, on the other hand. The results obtained are set out in Table I which illustrates the value of hydrolysis according to the invention.

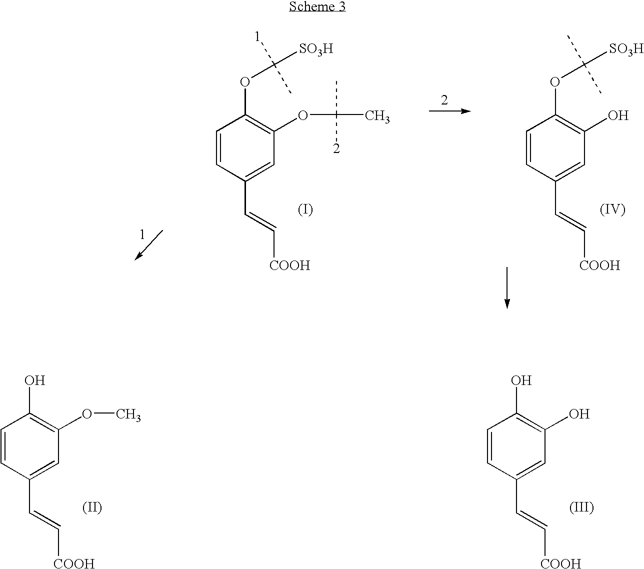

TABLE I

Antiradical activity
relative to ferulic acid
after hydrolysis

| | without | with |
|---|---|---|
| Product | hydrolysis | hydrolysis |
| trans-ferulic acid (II) | 1.00 | 2.05 |
| mixture of cis/trans | 0.95 | 1.95 |
| isomers (47/53 wt./wt.) of | | |
| ferulic acid | | |
| trans-caffeic acid (III) | 2.06 | 2.06 |
| sulfated ferulic acid (I) | 0.01 | 2.06 |
| Trolox ® (comparison | 0.53 | 0.55 |
| product) | | |

Similar results are obtained when sulfated ferulic acid (I) is replaced by an oside of ferulic acid, in particular glucuronide, and hydrolysis is performed with an appropriate osidase instead of the sulfatase.

Polyphenols of the flavonoid family, glycones having a greater antiradical reserve, due to the presence of one or more hydrolysable oside residues, than the aglycones, in particular cyanidine, genisteine, procyanidine, catechin and the glycones thereof are also suitable.

The method of the invention has been standardised for use with whole blood, erythrocytes or blood plasma as the second cellular material. In a reservoir, in particular a macroplate well, a dilute solution (S) of:
blood (60 μl) in dilution solution (1440 μl), erythrocytes prediluted to 50% (60 μl) in dilution solution (1440 μl), or plasma (60 μl) in dilution solution (1440 μl),
   is prepared and 50 μl of said solution (S) are transferred into the wells of a microplate containing a dilution solution (220 μl), the resultant mixture is enzymatically hydrolysed, washing is performed if necessary, a free radical generator (AAPH) is introduced and then release of the free radicals is initiated at 37° C. The T½ value and the other parameters stated above are then determined. Intermediate washing after hydrolysis may be omitted because it is not essential.

The sample to be tested (50 μl), which contains or is associated with whole blood, with erythrocytes or, respectively, with plasma and if necessary is diluted, is introduced into the dilution solution (220 μl) placed in the wells of a microplate. Hydrolysis is performed, the free radical generator is introduced, release of the free radicals is initiated and the T½ value and said other parameters are determined according to the methods stated above.

In practice, it is recommended to use the following quantities of enzymes, where U denotes the international unit of the enzyme in question:
β-glucosidase at a final concentration of 11.11 U/ml (3 U/well),
sulfatase at a final concentration of 3 U/ml (0.8 U/well), and/or
β-glucuronidase at a final concentration of 2222.22 U/ml (600 U/well).
   Other advantages and characteristics of the invention will be better understood on reading the Examples and tests stated hereafter. These are, of course, not limiting, but provided by way of illustration.

### EXAMPLE 1

### Quercetin/Isoquercitrin

Resistance to radical attack (by means of the above-stated AAPH) in whole control ewe's blood was assessed in the presence of an aglycone form of flavonoid, quercetin, and the glycone form thereof, isoquercitrin, without the hydrolysis of step (a), on the one hand, and with said hydrolysis, on the other hand.

FIG. 3 shows the results obtained with doses of quercetin or isoquercitrin rising from 20 μM to 100 μM without prior hydrolysis.

FIG. 4, plotted on the basis of the results of FIG. 3, shows that quercetin has antioxidant power greater than that of isoquercitrin when the T½ value is assessed as a function of concentration.

FIG. 5 shows that, after hydrolysis by means of an enzymatic mixture of β-glucosidase (3 U/well)+sulfatase (0.8 U/well)+β-glucuronidase (600 U/well), quercetin and isoquercitrin have identical antiradical resistance. The isoquercitrin has thus indeed been hydrolysed into quercetin by the enzymatic mixture, and release of the phenol function generates an available antiradical activity relative to an initially unavailable activity held “in reserve”.

FIG. 5 furthermore shows the activity of the mixture of enzymes [β-glucosidase (3 U/well)+sulfatase (0.8 U/well)+β-glucuronidase (600 U/well)] when the concentration of isoquercitrin or quercetin is zero.

### EXAMPLE 2

### Tests on Plasma

Experiments were performed, according to mode (III) above, on frozen plasma from healthy ewes (A) or ewes suffering from scrapie (B), said plasma acting as a biological agent in association with inspected erythrocytes, without and with prior enzymatic hydrolysis by means of β-glucosidase at increasing doses of 0 U/ml, 1.11 U/ml, 5.55 U/ml and 11.1 U/ml.

The results obtained are shown in Table II hereafter (mean of five tests per batch, with 3 batches from ewes A, and 3 batches from ewes B; SD=standard deviation from the mean; CV=coefficient of variation).

It is observed that, without prior hydrolysis, the ewes B have better antiradical resistance. This an anomaly due to a release of reserves in response to the oxidative aggression associated with the pathological condition.

After prior hydrolysis with a dose de 1.11 U/ml of β-glucosidase, it is noted that there is no appreciable difference relative to the tests without hydrolysis.

After prior hydrolysis with a dose of 5.55 U/ml of β-glucosidase, it is noted that the overall antiradical potential of the ewes A is slightly less than that of the ewes B. This means that the antiradical reserves have not yet been totally released by hydrolysis.

On the other hand, after prior hydrolysis with a dose of 11.1 U/ml of β-glucosidase, it will be noted that the overall antiradical potential of the ewes A is greater than that of the ewes B: the antiradical reserve has finally been mobilised by hydrolysis.

TABLE II
Tests on ewe plasma (dilution 1/135)

| | | | | |
|---|---|---|---|---|
| | Vₘₐₓ | Lag time | | EAR |
| Batches | (mAU/min) | (min) | (min) | (eq.mM) |

without prior hydrolysis (glucosidase: 0 U/ml)

| | | | | |
|---|---|---|---|---|
| B | −6.55 | 76.26 | 203.57 | 35.49 |
| (SD) | (0.24) | (4.09) | (7.32) | (2.45) |
| (CV) | (3.67) | (5.36) | (3.60) | (6.89) |
| A | −7.82 | 77.21 | 186.35 | 29.73 |
| (SD) | (1.26) | (2.50) | (16.18) | (5.41) |
| (CV) | (16.11) | (3.24) | (8.68) | (1.18) |

with prior hydrolysis (glucosidase: 1.11 U/ml)

| | | | | |
|---|---|---|---|---|
| B | −6.91 | 98.23 | 216.51 | 39.81 |
| (SD) | (1.22) | (22.30) | (3.59) | (1.20) |
| (CV) | (17.70) | (22.71) | (1.66) | (3.02) |
| A | −7.33 | 87.11 | 198.48 | 33.79 |
| (SD) | (0.55) | (9.49) | (11.95) | (3.99) |
| (CV) | (7.47) | (10.90) | (6.02) | (11.82) |

with prior hydrolysis (glucosidase: 5.55 U/ml)

| | | | | |
|---|---|---|---|---|
| B | −8.06 | 197.49 | 281.37 | 61.49 |
| (SD) | (1.15) | (11.08) | (1.74) | (0.58) |
| (CV) | (14.28) | (5.61) | (0.62) | (0.95) |
| A | −7.73 | 186.05 | 274.64 | 59.24 |
| (SD) | (0.60) | (13.80) | (8.92) | (2.98) |
| (CV) | (7.71) | (7.42) | (3.25) | (5.03) |

with prior hydrolysis (glucosidase: 11.1 U/ml)

| | | | | |
|---|---|---|---|---|
| B | −9.76 | 260.01 | 318.72 | 73.97 |
| (SD) | (0.56) | (11.91) | (0.44) | (0.15) |
| (CV | (5.69) | (4.58) | (0.14) | (0.20) |
| A | −13.00 | 308.91 | 328.84 | 77.35 |
| (SD) | (3.87) | (40.97) | (13.17) | (4.40) |
| (CV) | (29.74) | (13.26) | (4.00) | (5.69) |

### EXAMPLE 3

### Tests on Vines

Two vines were used, one producing red dessert grapes, the other black dessert grapes. Each vine was divided into two batches, the first receiving a foliar fertilizer (containing polyphenols) by spraying, the second being untreated and acting as control. During growth, comminuted leaves, comminuted shoots and pressed fruit juice, each suspended or diluted in a dilution buffer, were subjected to the method for determining antiradical defence potential according to the invention. It was noted that the treated vines exhibited greater potential than the untreated vines. The results obtained with grape juice are shown in the FIG. 6.

### EXAMPLE 4

### Tests on Pigs

Tests were carried out on a batch of 15 pigs, using animals considered to be healthy, the enzymatic hydrolysis being carried out (to save time) during exposure to the field of free radicals released at 37° C. from the above-stated free radical generator AAPH.

The results obtained are shown in Table III below, which shows values T½, N′ (normal range, i.e. range N in the absence of hydrolysis and ranges Nh, in this case localised, with hydrolysis), latency time (“lag time”) and maximum rate of radical lysis (Vₘₐₓ), as a function of enzymatic hydrolysis [sulfatase (at 3 U/ml) or β-glucuronidase (at 2500 U/ml)].

### EXAMPLE 5

### Tests in Humans

Tests were carried out on a group of 97 human subjects considered to be healthy (adult men and women) and a group of 92 diabetic human subjects (adult men and women). Whole blood from each of the subjects of the two groups was subjected (1) in a first series of experiments to exposure to the field of free radicals generated at 37° C. from the above-stated free radical generator AAPH, and (2) in a second series of experiments to enzymatic hydrolysis [with β-glucosidase (3 U/well), sulfatase (0.8 U/well), β-glucuronidase (600 U/well) or the above-stated ternary mixture] implemented during said exposure to the same field of free radicals, each group acting as its own control.

The results obtained with hydrolysis by means of β-glucosidase (3 U/well) are shown in Table IV below, which reveals that in the healthy subjects the mean T½ value changes from 92.90 min without hydrolysis to 263.85 min with hydrolysis (i.e. a released reserve of 184%), whereas in the diabetics the mean T½ value changes from 99.54 min without hydrolysis to 215.94 min with hydrolysis (i.e. a released reserve of only 116%). The results obtained on human plasma are similar.

TABLE III
Tests in pigs

| | T½ | N′ | Lag time | Vₘₐₓ |
|---|---|---|---|---|
| Sample | (min) | (min) | (min) | (mAU/min) |
| whole blood | 92.40 | 84-100 | 69.29 | −21.85 |
| whole blood + sulfatase | 100.70 | 91-110 | 74.63 | −20.24 |
| whole blood + β- | 158.69 | 135-181 | 122.87 | −9.24 |
| glucuronidase | | | | |
| erythrocytes | 69.19 | 62-76 | 55.16 | −28.47 |
| erythrocytes + sulfatase | 80.96 | 75-88 | 62.23 | −23.51 |
| erythrocytes + β- | 169.35 | 158-180 | 149.92 | −12.02 |
| glucuronidase | | | | |

TABLE IV
Tests in humans

| | T½ (min) | |
|---|---|---|
| | healthy | T½ (min) |
| Sample | subjects | diabetic subjects |
| whole blood | 92.90 | 99.54 |
| whole blood + β- | 263.85 | 215.94 |
| glucosidase | | |

This set of tests shows that the method of the invention makes it possible to monitor the growth of animals and plants, in particular that of strategic plants such as cereals (in particular wheat, maize and rice), oil crops (in particular soya, peanut and rapeseed), vines and cotton.

Said method also makes it possible to monitor changes in the state of health of livestock. Thanks to said method, it is possible to determine, species by species, a normal range (i.e. a range of normal values) below which the animals should be carefully monitored with the aim, if necessary, of screening out or withdrawing their tissues from consumption. In application of said results, it is more particularly recommended to feed those animals to be sent to the abattoir with a diet supplemented with antiradical substances, in particular sulfated ferulic acid, the osides of said ferulic acid and polyphenols. Such a diet may comprise as a nutritional supplement a cocoa extract with a high polyphenol content, or even a composition based on cocoa pod cortex supplemented with cocoa pod mucilage and/or cocoa bean juice.

Sulfated ferulic acid, the osides of said ferulic acid (in particular glucuronide), polyphenols, said cocoa extract and/or said composition based on cocoa pod cortex are also of use in humans for treating and preventing stress states.

### EXAMPLE 6

### Use of a Colorant

The above-stated blue (methylene blue) and red (OPC extract) colorants were used with the aim of standardising the method of the invention implemented without cellular material. The results shown hereafter are the mean of 6 tests.

In a first series of experiments, it was verified that, at time T=0 (T₀) when release of the free radicals is not induced, the OD (at 260 nm) of the blue colorant as a function of its concentration is linear, whether used alone (curve 1 of FIG. 7a) or in association with the FRG (curve 2 of FIG. 7a). It will be noted that curve 1 is linear:
y=0.09986x+0.03278 r²=0.9999
and that curve 2 is likewise linear:
y=0.0964x+0.0431 r²=0.9996

For the red colorant, at time T₀ the variation in OD (at 450 nm) is linear, the curves 1 (without FRG) and 2 (with FRG) of FIG. 7b being nearly superimposed on one another. Curve 1 is:
y=0.03268x+0.0608 r²=0.9997
and curve 2 is:
y=0.3264x+0.0511 r²=0.9999

In a second series of experiments, the effect of the generator on each of the two colorants over time was evaluated. The OD of the blue colorant measured at 620 nm declines whereas that of the red colorant measured at 450 nm rises. See FIGS. 7c (blue colorant) and 7d (red colorant).

In a third series of experiments, the variation in OD of the blue colorant (FIG. 7e), which is rising, and that of the red colorant (FIG. 7f) which is rising, was investigated as a function of initial FRG concentration at time T₀ then after release of the free radicals.

### EXAMPLE 7

### Study of Four Lines of Gallinaceae

The antiradical resistance of whole blood, erythrocytes and of plasma together with the release of reserves R1 (hydrolysis by means of glucosidase), R2 (hydrolysis by means of sulfatase) and R3 (hydrolysis by means of glucuronidase) was studied on four lines of chickens (A1, A2, B1 and B2) of different breeds: broiler chickens (batches A1 and A2) and laying chickens (i.e. chickens for producing eggs; batches B1 and B2), each batch comprising twelve animals.

The results obtained, stated as the mean and standard deviation (SD), are set out in Table V hereafter and in FIGS. 8a to 8i.

This set of results shows that the study of the variation in antiradical defence reserves in animals makes it possible to target performance criteria, such as growth, weight, reproductive performance and the quality of production in particular with regard to genetic traits governing resistance to stress and/or to disease.

TABLE V

| | | Whole | | | | | | | | | | |
|---|---|---|---|---|---|---|---|---|---|---|---|---|
| | | blood | Erythrocytes | | Whole blood | | | | Erythrocytes | | | |
| | | T½ | T½ | Plasma | R1 | R2 | R3 | | R1 | R2 | R3 | |
| Groups | | (min) | (min) | T½ (a) | (b) | (b) | (b) | Sum (b) | (c) | (c) | (c) | Sum (c) % |
| A1 | MEAN | 75.93 | 70.02 | 7.77 | 27.63 | 8.00 | 92.70 | 128.33 | 107.21 | 33.71 | 162.79 | 303.71 |
| | SD | 3.72 | 2.93 | 4.87 | 7.22 | 4.10 | 9.96 | 17.79 | 12.32 | 4.26 | 15.61 | 21.25 |
| A2 | MEAN | 85.08 | 73.73 | 12.81 | 31.29 | 10.55 | 90.55 | 132.39 | 104.62 | 36.11 | 158.00 | 298.72 |
| | SD | 8.71 | 2.98 | 6.03 | 10.93 | 4.69 | 26.38 | 31.47 | 24.97 | 9.41 | 28.28 | 57.44 |
| B1 | MEAN | 74.20 | 62.55 | 15.30 | 13.69 | 3.13 | 36.48 | 53.30 | 61.49 | 18.66 | 111.62 | 191.78 |
| | SD | 9.20 | 5.29 | 4.74 | 5.36 | 1.98 | 35.99 | 39.47 | 8.74 | 2.57 | 38.69 | 42.92 |
| B2 | MEAN | 79.94 | 69.87 | 12.32 | 21.07 | 3.76 | 56.65 | 81.48 | 98.42 | 19.54 | 144.17 | 262.14 |
| | SD | 8.48 | 6.03 | 4.96 | 6.33 | 2.15 | 25.27 | 30.16 | 25.92 | 2.92 | 28.11 | 40.14 |

Notes
(a) for plasma, the T½ value is expressed in % relative to the T½ value for whole blood
(b) for R1, R2, R3 and the sum thereof, the T½ value for whole blood is expressed in % relative to the T½ value of the whole blood of the controls
(c) for R1, R2, R3 and the sum thereof, the T½ value for the erythrocytes is expressed in % relative to the T½ value of the erythrocytes of the controls

### EXAMPLE 8

### Study on Rats

Tests were carried out on normal rats taken as controls and on rats made diabetic by treatment with streptozotocin. The results obtained are shown in FIGS. 9a to 9d.

The free plasma antiradical defences of the rats made diabetic by streptozotocin have been induced (plasma defence=whole blood defence−erythrocytes defence). This induction occurs to the detriment of the released circulating reserves in particular for the reserves R1 which drop by more than one third, the T½ value remaining practically identical in the erythrocytes of the controls and of the diabetic subjects since this is the onset of diabetes. It will be noted that the reserves R1 and R2 in the erythrocytes remain almost identical between the controls and the diabetic subjects and that the intracellular reserves R3 increase to protect the erythrocytes to the detriment of plasma reserves.

### EXAMPLE 9

### Monitoring of the Ageing of Wine

Tests were carried out on five different wines, wine 1 being more than 40 years old, wine 2 being 14 years younger than wine 1, wines 3, 4 and 5 originating from the same vineyard plot for the years 1992, 1997 and 2000 respectively. Ageing is accelerated either in the normal manner by opening each bottle and allowing oxidation to proceed, or artificially by means of free radicals.

The results obtained are shown in FIGS. 10a (prior art KRL test) and 10b (in accordance with the method of the invention). Curve 10a shows that wine 2 is totally maderised since its oxidation resistance time is too short. FIG. 10b shows that wine 5 will age more quickly than wine 4, on the one hand, and that wine 1 (a masterpiece) may be enjoyed from now until several years hence.

In a nutshell, the method of the invention

(1) is particularly well suited to the in vitro monitoring of the state of health of humans, animals and plants, with regard to their nutrition (and/or to in vitro monitoring of the nutritional input provided to them), to the management of stress and to slowing ageing, on the one hand, and to in vitro monitoring of the genetic potential of animals and plants, for the purposes of selecting them, on the other hand, and

(2) reveals that antiradical substances are essential for preventing spongiform encephalitis (BSE, OSE or CJD) and autoimmune diseases (in particular Alzheimer's disease, Parkinson's disease and allergies) which disrupt the control system of overall antiradical defences.

Accordingly, the method of the invention is particularly useful for assessing, in antiradical terms, the nutritional worth or value of foodstuffs, food or nutritional supplements (“nutraceutics”) and beverages, on the one hand, and for monitoring each step in the industrial processing of said foodstuffs, food supplements and beverages with the aim of preserving the health of humans, animals or plants and of respecting the environment.

It is thus recommended according to the invention, on the one hand, to use the present method for the in vitro determination of the overall antiradical defence potential,

said use being characterised in that it comprises the performance of said method for in vitro monitoring of the growth of animals and plants and/or in vitro monitoring of the nutritional input provided to them, and, on the other hand, to use said method for the in vitro determination of the overall antiradical defence potential, said use being characterised in that it comprises the in vitro performance

of said method in humans for monitoring the state of health, managing stress and/or monitoring ageing.

1. A method for the in vitro determination of the overall antiradical defence potential of a living organism or of a physical, chemical or biological agent, said method, involving the use of free radicals as a means for inducing cell lysis followed by evaluation of said cell lysis, the method comprising hydrolysis of a cellular material before or during release, in a resultant reaction medium, of free radicals from a free radical generator.

2. A method according to claim 1 for the in vitro determination of the overall antiradical defence potential of a living organism or of a physical, chemical or biological agent, involving, the use of free radicals as a means for inducing cell lysis, the method comprising the following steps:
(a) hydrolysis of a sample
(i) of a first cellular material from a living organism, said first cellular material being a cellular tissue, cells or a cell fragment, or, respectively,
(ii) of a second cellular material associated with a physical, chemical or biological agent, said second cellular material being a reference product which is a cellular tissue, cells, a cell fragment or a synthetic wall containing liposomes,
(b) during or after said hydrolysis, bringing said sample into contact with a free radical generator,
(c) inducing release of the free radicals from said free radical generator, and
(d) monitoring lysis, by optical measurement, of the first or, respectively, second cellular material, relative to a control sample, in order to assess the overall antiradical potential of said living organism or, respectively, of said physical, chemical or biological agent to be tested.

3. The method according to claim 2, wherein, in step (d), the T½ value is determined which corresponds to the lysis of 50% of the first cellular material or the second cellular material and which expresses the overall antiradical potential of said first cellular material or of said cellular material associated with said physical, chemical or biological agent.

4. The method according to claim 2 wherein said first cellular material is whole blood.

5. The method according to claim 2 wherein said second cellular material is whole blood or erythrocytes.

6. The method according to claim 2 wherein said biological agent is plasma from a human subject or from a warm-blooded animal.

7. The method according to claim 2, wherein the hydrolysis of stage (a) is (i) enzymatic hydrolysis, (ii) acid or alkaline hydrolysis, or (iii) cleaving by means of a physical agent, in particular radiation.

8. The method according to claim 2, wherein the second cellular material is replaced by a colorant which is degradable by free radicals.

9. The method according to claim 8 for assessing the antiradical resistance of a physical, chemical or biological agent, said method being wherein said method comprises the following steps:
(a) hydrolysis of a sample
of a colorant which is degradable by free radicals and which is associated with a physical, chemical or biological agent,
(b) during or after said hydrolysis, bringing said sample into contact with a free radical generator,
(c) inducing release of the free radicals from said free radical generator, and
(d) monitoring the lysis, by optical measurement, of said colorant relative to a control sample, in order to assess the overall antiradical potential of said sample containing said physical, chemical or biological agent to be tested.

10. The method according to claim 8, wherein said colorant is methylene blue or the red colorant extracted from oligoanthocyanidin.

11. Use of the method according to claim 1, wherein the use comprises the performance of said method for in vitro monitoring of the growth of animals and plants and/or in vitro monitoring of the nutritional input provided to them.

12. Use of the method according to claim 1, wherein the use comprises the in vitro performance of said method in humans for monitoring the state of health, managing stress and/or monitoring ageing.

13. Use according to claim 11, wherein the antiradical substance is sulfated ferulic acid or an osides of ferulic acid.

14. Use of an antiradical substance for the preparation of a medicine, wherein an antiradical substance is used for the preparation of a medicine intended for use in human or veterinary therapeutics against spongiform encephalitis, wherein said encephalitis is selelected from the group consisting of bovine spongiform encephalitis (BSE), ovine spongiform encephalitis (OSE) and human spongiform encephalitis (CJD).

15. Use according to claim 11 for monitoring the ageing of wine.
